(19) United States
(12) Patent Application Publication
(10) Pub. No.: US 2012/0282334 A1
(43) Pub. Date: Nov. 8, 2012

(54) PROCESS FOR PREPARING AJOENE FROM GARLIC

(75) Inventors: Dong Bin Shin, Gyeonggi-do (KR); Mi Young Yoo, Gyeonggi-do (KR); Ho Moon Seog, Gyeonggi-do (KR); Jae Ho Ha, Gyeonggi-do (KR); Sang Hee Lee, Seoul (KR); Yong Sun Cho, Seoul (KR)

(73) Assignee: KOREA FOOD RESEARCH INSTITUTE, Gyeonggi-do (KR)

(21) Appl. No.: 13/290,361

(22) Filed: Nov. 7, 2011

(30) Foreign Application Priority Data
May 2, 2011 (KR) 10-2011-0041443

(51) Int. Cl. A61K 9/48; A61J 3/07; C07C 323/65; C07C 319/28; A61K 31/105

(52) U.S. Cl. 424/456; 568/22; 514/707; 427/2.22

(57) ABSTRACT

Disclosed is a method for preparing ajoene including: (a) obtaining garlic pulp or garlic extract from garlic; (b) mixing the garlic pulp or garlic extract with animal lipid to form a reaction mixture; and (c) heating the reaction mixture to obtain ajoene. The ajoene prepared according to the present disclosure may be encapsulated with gelatin. According to the present disclosure, ajoene can be prepared with a yield 2 times or more than that of the existing methods. Thus, ajoene can be provided as active ingredient for foods and medicines more cost-effectively. Also, the production protocol according to the present disclosure is suitable for large-scale production of ajoene.

### TECHNICAL FIELD

The present disclosure relates to a method for preparing ajoene from garlic using animal lipid, particularly milk fat.

### BACKGROUND

Garlic (Allium sativum L.) is a bulbous plant belonging to the genus Allium of the family Liliaceae. Recently, many researchers have reported various pharmacological and physiological activities including anticancer effect, preventing effect on the blood circulation disorder and antioxidant activity. The main ingredients of garlic exhibiting such pharmacological and physiological activities are known as sulfur compounds, including allicin which is produced by the action of the enzyme alliinase from S-allyl-L-cysteine sulfoxide, an S-alk(en)yl-L-cysteine sulfoxide also known as alliin, and ajoenes and vinyldithiins which are produced from polymerization or degradation of allicin (Lawson et al. Planta. Med. 57: 363-370 (1991)). Ajoene (E,Z-4,5,9-trithiadodeca-1,6,11-triene 9-oxide) is produced from pure allicin and is chemically more stable than allicin. Ajoene exists in two isomer forms, either as Z-ajoene (cis form) of the chemical formula 1 or as E-ajoene (trans form) of the chemical formula 2.

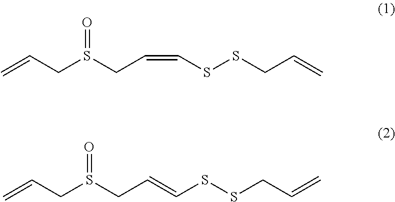

Block et al. first reported that ajoene is produced from chopped garlic in organic solvent or edible oil (J. Am. Chem. Soc. 106: 8295-8296 (1984)). According to Block's study, ajoene inhibits all the factors involved in platelet aggregation and the effect is comparable to that of aspirin (Sci. Am. 252: 94-99 (1985)).

Ajoene's biological activities include prevention of thrombosis, antifungal activity (Stenier M. et al. J. Nutr. 131: 980S-984S (2001)), and reduction of cholesterol biosynthesis through inhibition of 3-hydroxy-3-methylglutaryl-CoA (HMG-CoA) reductase (Rolf et al. Biochimica et Biophysica Acta. 1213: 57-62 (1994)). It is reported that garlic affects lipid metabolism in the body and is effective in reducing blood lipid level as well as in preventing arteriosclerosis, thrombosis and blood circulation disorder caused by vasoconstriction, which are the direct cause of hypertension, heart disease, stroke, etc.

By elucidating the mechanism by which ajoene prevents platelet aggregation, Apitz-Castro et al. (Biochim. Biophys. Acta. 1094: 269-280 (1991)) showed the possibility of ajoene for the treatment of cardiovascular diseases such as pulmonary embolism, arteriosclerosis, hyperlipidemia, etc. Also, ajoene has an effect such as inhibiting the binding of carcinogen aflatoxin B and DNA (Tadi et al. Cancer Letters 59: 89-94 (1991)) and cytotoxicity for tumor cells (Scharfenberg et al. Cancer Letters 53: 103-108 (1990)) was studied.

Also, ajoene is reported to have antifungal/antibacterial activity (Yoshida et al. Appl. Environ. Microbiol. 53: 615-617 (1987)) and antiviral activity (Werber et al. Planta Med. 58: 417-423 (1992)) and is effective for AIDS (acquired immune deficiency syndrome) (Tatarintsev et al. Int. Conf. AIDS 8: 19-24). Ajoene exhibits an effect comparable to that of the antifungal/antibacterial agent, 5-fluorocytosine.

Ajoene is not found in garlic itself or generally processed garlic such seasoned garlic or dried garlic. It is formed in general when garlic is mixed with a less polar organic solvent or oil/fat. The production amount varies greatly depending on the polarity of the solvent, fatty acid composition and reaction conditions (temperature and time).

Lawson obtained 60-148 μg/g of ajoene from the garlic juice obtained by adding garlic in vegetable oil and reported that E-ajoene is produced in general twice or more than Z-ajoene.

Japanese Patent Application Publication No. 62-129224 discloses a procedure whereby garlic is mixed with an organic solvent of pH 2-6 such as ethyl alcohol and methyl alcohol and heated to 40-90° C. to extract ajoene. This method requires a large amount of garlic because only about 900 μg of ajoene is extracted from 1 kg of garlic. Such a low efficiency is not enough for application to food or medicine.

In U.S. Pat. No. 5,612,077, Takayoshi Hibi obtained 303-404 mg of ajoene from 1 kg of garlic by mashing garlic in edible oil.

### SUMMARY

The inventors of the present disclosure have made efforts to develop an optimized process for producing the functional or medical active ingredient ajoene from garlic. As a result, they have developed a process for preparing ajoene using animal lipid, particularly milk fat, ensuring preparation of ajoene at a yield of at least two times better than that of the existing methods.

The present disclosure is directed to providing a method for preparing ajoene.

The present disclosure is also directed to providing ajoene prepared by the method according to the present disclosure.

In one general aspect, the present disclosure provides a method for preparing ajoene including: (a) obtaining garlic pulp or garlic extract (garlic juice) from garlic; (b) mixing the garlic pulp or garlic extract with animal lipid to form a reaction mixture; and (c) heating the reaction mixture under a specific condition to obtain ajoene.

In another general aspect, the present disclosure provides ajoene prepared by the above method.

Other features and aspects will be apparent from the following detailed description, the drawings, and the claims.

### BRIEF DESCRIPTION OF THE DRAWINGS

The above and other objects, features and advantages of the present disclosure will become apparent from the following description of certain exemplary embodiments given in conjunction with the accompanying drawings, in which:

FIG. 1 shows a process of preparing ajoene from garlic established by the inventors of the present disclosure (In the step 1, garlic pulp or garlic juice is prepared from garlic. In the step 2, garlic is mixed with animal lipid to maximize the production of ajoene. In the step 3, ajoene is extracted, purified and stabilized.);

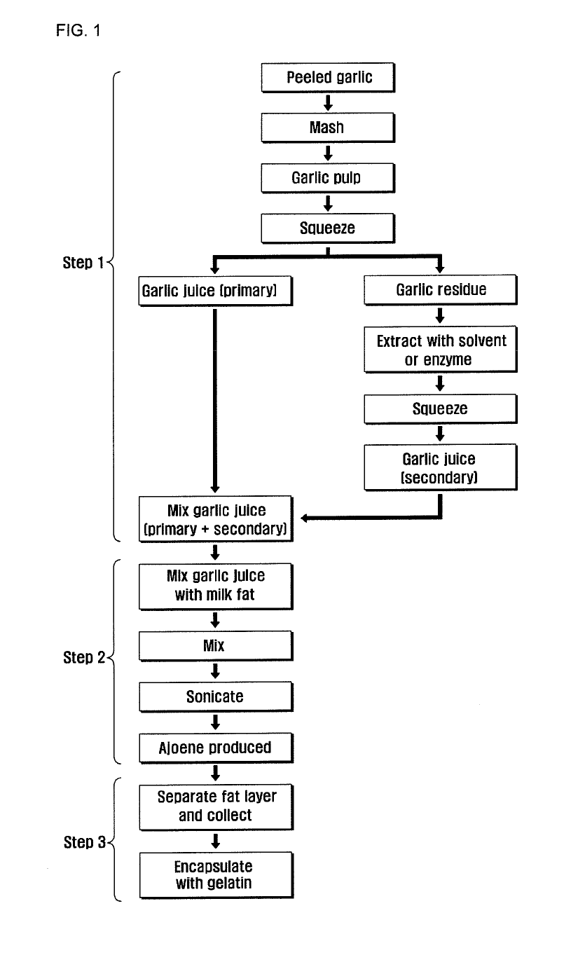

FIG. 2 shows chromatograms of ajoene compounds using high-performance liquid chromatography (HPLC) established by the inventors of the present disclosure [(A): ajoene isomers standard, (B): reaction product of animal lipid and garlic juice, (C): blank]; and

FIG. 3 shows response surface plots for optimizing preparation of ajoene from garlic juice [(A): response surface plot of reaction temperature X₁ and oil volume X₃, (B): response surface plot of reaction time X₂ and reaction temperature X₁, (C): response surface plot of oil volume X₃ and reaction time X₂].

### DETAILED DESCRIPTION OF EMBODIMENTS

The advantages, features and aspects of the present disclosure will become apparent from the following description of the embodiments with reference to the accompanying drawings, which is set forth hereinafter. The present disclosure may, however, be embodied in different forms and should not be construed as limited to the embodiments set forth herein. Rather, these embodiments are provided so that this disclosure will be thorough and complete, and will fully convey the scope of the present disclosure to those skilled in the art. The terminology used herein is for the purpose of describing particular embodiments only and is not intended to be limiting of the example embodiments. As used herein, the singular forms “a”, “an” and “the” are intended to include the plural forms as well, unless the context clearly indicates otherwise. It will be further understood that the terms “comprises” and/or “comprising”, when used in this specification, specify the presence of stated features, integers, steps, operations, elements, and/or components, but do not preclude the presence or addition of one or more other features, integers, steps, operations, elements, components, and/or groups thereof.

Hereinafter, exemplary embodiments will be described in detail with reference to the accompanying drawings.

The inventors of the present disclosure have made efforts to develop an optimized process for producing the functional or medical active ingredient ajoene from garlic with high yield. As a result, they have developed a process for preparing ajoene using animal lipid, particularly milk fat, ensuring preparation of ajoene at a yield of at least two times better than that of the existing methods.

A method for preparing ajoene according to the present disclosure will be described in detail:

Step (a): Preparation of Garlic Pulp or Garlic Juice

First, garlic pulp is obtained from garlic or garlic juice is obtained from garlic pulp. The garlic pulp or garlic juice may be obtained according to various methods known in the art. For example, the garlic pulp may be prepared by various physical processes. The garlic juice may be obtained by combining the garlic juice obtained by physical mashing and primary squeezing with an extract obtained by extracting the residue with an extraction solvent (e.g., water or ethanol). Instead of the solvent such as water or ethanol, a pectin- or cellulose-degrading enzyme (e.g., pectinase or cellulase) solution may be added to the residue.

Specifically, the garlic pulp may be 20 mesh or less. The garlic juice may be one obtained by physical mashing or physical mashing and squeezing or one obtained by combining the garlic juice with an extract obtained by extracting with an extraction solvent.

The garlic juice obtained by the above method contains allicin as a starting material for preparing ajoene.

Step (b): Formation of Reaction Mixture of Garlic Juice (or Garlic Pulp) and Milk Fat

Then, the garlic juice (or garlic pulp) is mixed with animal lipid to form a reaction mixture.

One of the features of the present disclosure is to use milk fat to prepare ajoene. Although plant oil such as soybean oil, rice bran oil and olive oil has been commonly used in the preparation of ajoene from garlic, the milk fat with a significantly different fatty acid composition is used in the present disclosure.

As used herein, the term “milk fat” refers to a lipid originating from animal, such as cow, goat, sheep and horse, i.e. a biomolecule including fat, sterol, monoglyceride, diglyceride, triglyceride, phospholipid and fatty acid.

In an exemplary embodiment of the present disclosure, the milk fat is cream or butter, more specifically butter, comprising 30% or more of fat.

Specifically, the fatty acid included in the milk fat includes saturated fatty acids and unsaturated fatty acids as follows (see National Research Council, 1976, Fat Content and Composition of Animal Products, Printing and Publishing Office, National Academy of Science, ISBN 0-309-02440-4; p. 203)—saturated fatty acids: lauric acid 2-5%, myristic acid 5-20%, palmitic acid 20-40% and stearic acid 5-20%; and unsaturated fatty acids: oleic acid 15-30%, palmitoleic acid 1-8%, linoleic acid 1-6% and linolenic acid 0.5-3%. More specifically, the fatty acid composition of the milk fat may be, saturated fatty acids: lauric acid 2.5-4%, myristic acid 10-15%, palmitic acid 28-35% and stearic acid 8-13%; and unsaturated fatty acids: oleic acid 20-27%, palmitoleic acid 2-6%, linoleic acid 2-5% and linolenic acid 0.8-2%.

In an exemplary embodiment of the present disclosure, the weight ratio of the garlic juice to the milk fat in the reaction mixture of the step (b) is 1:2-1:8, more specifically 1:2-1:5, most specifically 1:2.8-1:4.

In an exemplary embodiment of the present disclosure, the method according to the present disclosure further comprises sonicating the reaction mixture with ultrasonic waves between the steps (b) and (c). The sonication using the ultrasonic waves may be performed using a sonicator.

Step (c): Preparation of Ajoene

Finally, the reaction mixture is heated to obtain ajoene.

Another feature of the present disclosure is to heat the reaction mixture to obtain ajoene. In an exemplary embodiment of the present disclosure, the step (c) is performed at 30-90° C., more specifically at 30-80° C., most specifically at 50-70° C.

In an exemplary embodiment of the present disclosure, the step (c) is performed for 1-6 hours, more specifically for 1-4 hours, most specifically for 1.5-2.5 hours.

Step (d): Extraction, Purification and Stabilization of Ajoene

In an exemplary embodiment of the present disclosure, the method according to the present disclosure further comprises, after the step (c), (d) purifying and encapsulating the ajoene with gelatin. More specifically, the ajoene formed in the oil layer is purified by extraction, for example, using a centrifuge and encapsulated with gelatin.

More specifically, after the reaction is completed, the reaction mixture is quickly cooled to room temperature and the oil layer is taken. The cooling is performed using a cooling system and the oil layer containing ajoene is taken, for example, using a centrifuge.

The ajoene existing in the oil layer is encapsulated with gelatin to prevent loss.

The term ‘gelatin’ refers to a protein obtained by extracting the natural protein collagen constituting the skin, tendon and cartilage of animal with hot water or treating (partially hydrolyzing) with dilute alkali or acid. Gelatin is a protein derived from the skin or bones of pig or cow, with a molecular weight of about 15,000-250,000. Collagen is rich in the connective tissues of animal, including skin, tendon, ligament and bone. Although gelatin is a protein derived from animal, it is classified as incomplete protein since it contains no tryptophan, which is an essential amino acid, and is deficient in many other essential amino acids. The capsule may be either a hard-shelled capsule or a soft-shelled capsule. The hard-shelled capsule is used for powdery or particulate, miniature pellets or tablets. The soft-shelled capsule is used for oils or active ingredients dissolved/suspended in oils. As an alternative of the gelatin, plant polysaccharides such as carrageenans, derivatives thereof, starches or modified cellulose are used.

According to the method of the present disclosure, ajoene can be prepared with significantly improved yield as compared to the existing art (e.g., U.S. Pat. No. 5,612,077 [symbol] ). Further, the ajoene prepared according to the method of the present disclosure has a very high Z/E ratio. For example, the ajoene prepared according to the method of the present disclosure exhibits a Z/E ratio of 10. Considering that Z-ajoene has higher biological activity than E-ajoene, the improvement of the Z/E ratio by the present disclosure is of great technical/industrial value.

### EXAMPLES

The examples and experiments will now be described. The following examples and experiments are for illustrative purposes only and not intended to limit the scope of this disclosure.

### Example 1

### Analysis of Ajoene Existing in Garlic-Fat Mixture

Peeled garlic harvested in 2010 was purchased for test. The purchased garlic was mashed with a mashing machine (Oscar, Kimhae, Korea) into garlic pulp and juice. Only the garlic juice was packed in vacuum film and stored at −80° C. for use as test sample.

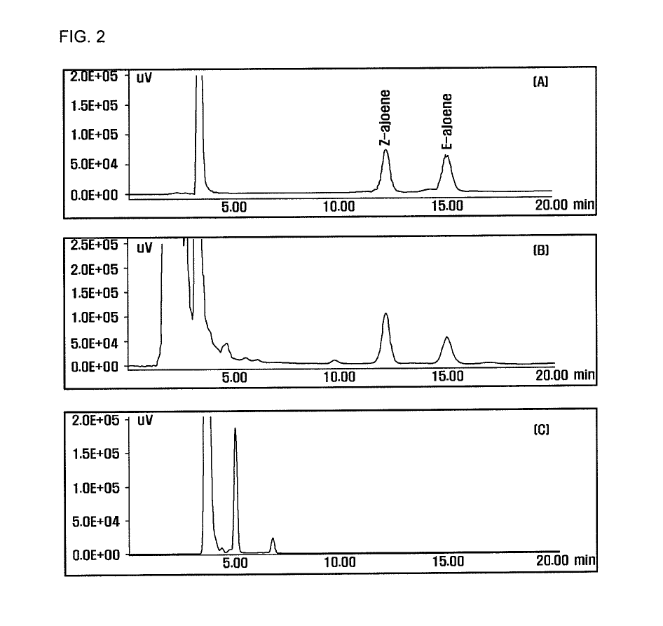

Garlic juice (5 g) was mixed with milk fat product containing 80% or more or fat of 1-5 times the amount of garlic juice. After vortexing for 1 minute, sonication was performed at 40° C. for 20 minutes using a sonicator. After making the garlic juice react with fat in a drying oven set at predetermined temperature, followed by centrifuging at 4000 rpm for 5 minutes, the supernatant was taken. After diluting 2-4 times with ethyl acetate, the content of ajoene was measured. The ajoene content was measured using a UV detector (JSACO, 2075 Plus) and an HPLC instrument (JSACO, pump: 2089 Plus, injector: 2057 Plus, column oven: 2067 Plus) equipped with a silica gel column (Agilent, 4.6×250 mm, 5 μm). The mobile phase for HPLC separation was hexane:2-propanol (85:15). The flow rate was 0.8 mL/min and the injection volume was 5 μL. The UV wavelength was 240 nm (FIG. 2).

### Example 2

### Optimization of Ajoene Preparation Condition by Response Surface Methodology

The animal fat butter that exhibited the most production of ajoene in the preliminary test was selected to explore the optimized condition for ajoene production. For this, the response surface methodology was employed and the central composite design (CCD) was selected as the experimental design. The three independent variable factors, reaction temperature X₁, reaction time X₂ and oil volume X₃, were coded as 5 levels (−2, −1, 0, 1, 2) (see Table 1). The dependent variable affected by the independent variables was ajoene production amount Y from the garlic-butter mixture. Experiment was performed under 17 conditions.

TABLE 1
Establishment of ajoene production conditions from garlic-butter mixture
by central composite design

| | | Levels | | | | |
|---|---|---|---|---|---|---|
| Symbols | Independent variable | −2 | −1 | 0 | 1 | 2 |
| X₁ | Reaction temperature (° C.) | 20 | 40 | 60 | 80 | 100 |
| X₂ | Reaction time (hr) | 1 | 2 | 4 | 6 | 8 |
| X₃ | Oil volume (times by weight) | 1 | 2 | 3 | 4 | 5 |

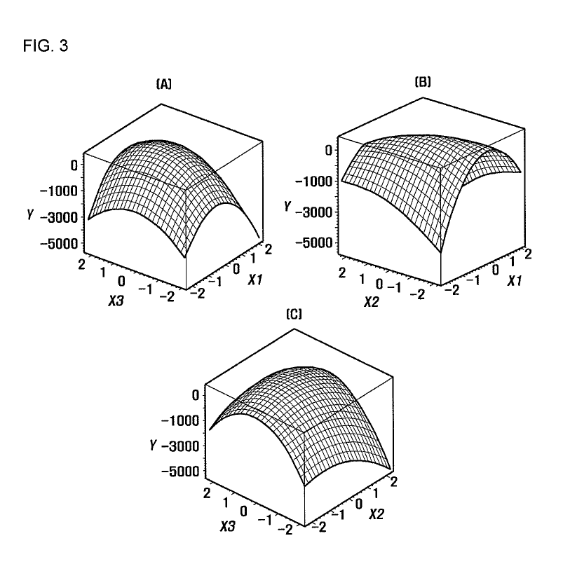

Statistical analysis was performed using SAS package ver. 8.2 (SAS Institute Inc., Cary, N.C., USA). The relationship between the independent variables and the dependent variable was expressed by a polynomial regression and 3-dimensional response surface plots were drawn based on the polynomial regression using Maple 7 (Waterloo Maple Inc., Canada) (FIG. 3).

Experimental result for the key independent variables, i.e. reaction temperature (20-100° C.), reaction time (1-8 hr) and oil volume (1-5 times), in the production of ajoene from the garlic-butter mixture is shown in Table 2. The ajoene content was 85.5-848.2 μg/g (garlic juice). As a result of response surface regression analysis (Table 3), R² was close to as 0.9189 and significance was acknowledged within 1% as 0.0045.

TABLE 2
Experimental result for different ajoene production conditions by central
composite design

| | Ajoene production condition | | | |
|---|---|---|---|---|
| | Reaction | Reaction | | |
| Run | temperature | time | Oil volume | Ajoene content |
| No. | (° C.) | (hr) | (times by weight) | (μg/g, garlic juice) |
| 1 | 40 (−1) | 2 (−1) | 2 (−1) | 469.8 |
| 2 | 40 (−1) | 2 (−1) | 4 (1) | 728.7 |
| 3 | 40 (−1) | 6 (1) | 2 (−1) | 437.4 |
| 4 | 40 (−1) | 6 (1) | 4 (1) | 633.6 |
| 5 | 80 (1) | 2 (−1) | 2 (−1) | 611.4 |
| 6 | 80 (1) | 2 (−1) | 4 (1) | 814.5 |
| 7 | 80 (1) | 6 (1) | 2 (−1) | 96.0 |
| 8 | 80 (1) | 6 (1) | 4 (1) | 620.3 |
| 9 | 60 (0) | 4 (0) | 3 (0) | 846.8 |
| 10 | 60 (0) | 4 (0) | 3 (0) | 848.2 |
| 11 | 60 (0) | 4 (0) | 3 (0) | 861.5 |
| 12 | 100 (2) | 4 (0) | 3 (0) | 85.5 |
| 13 | 20 (−2) | 4 (0) | 3 (0) | 494.7 |
| 14 | 60 (0) | 8 (2) | 3 (0) | 592.1 |
| 15 | 60 (0) | 1 (−2) | 3 (0) | 786.8 |
| 16 | 60 (0) | 4 (0) | 5 (2) | 726.6 |
| 17 | 60 (0) | 4 (0) | 1 (−2) | 312.2 |

TABLE 3
Polynomial for ajoene production from garlic-butter mixture by response
surface methodology

| Response | | | |
|---|---|---|---|
| value | Polynomial regression | R² | Significance |
| Ajoene | Y = 809.902496 − 154.602047X₁ − | 0.9189 | 0.0045 |
| content | 165.722972X₂ + 267.585826X₃ − | | |
| (μg/g, | 558.375375X₁₂ − 254.676991X₁X₂ − | | |
| garlic | 123.575677X₂₂ + 136.162237X₁X₃ + | | |
| juice) | 113.122560X₂X₃ − 329.021106X₃₂ | | |

When the change in ajoene content was analyzed from the 3-dimensional response surface for the experimental conditions designed by the central composite design, maximum points were observed as shown in FIG. 3.

The optimal condition for the preparation of ajoene from the garlic-butter mixture was reaction temperature=61.05° C., reaction time=2.56 hr and oil volume=3.63 times (Table 4). Under the optimal condition, ajoene could be produced from the garlic-butter mixture at 2 times higher concentration as compared to the existing method.

TABLE 4
Optimal condition expected by response surface methodology

| | | | Oil | | |
|---|---|---|---|---|---|
| | Reaction | Reaction | volume | | |
| Response | temperature | time | times by | | |
| value | ° C. | hr | weight | Maximum | Type |
| Ajoene | 61.05 | 2.56 | 3.63 | 896.08 | Maximum |
| content | | | | | |
| (μg/g, | | | | | |
| garlic | | | | | |
| juice) | | | | | |

As seen from Table 5, reaction temperature and oil volume had larger effect on the ajoene production than reaction time.

TABLE 5
Analysis of variance (significance) for ajoene content regression model

| | F-values | | |
|---|---|---|---|
| Response | Reaction temperature | Reaction time | Oil volume |
| value | ° C. | hr | times by weight |
| Ajoene | 11.10** | 3.95* | 9.10** |
| content | | | |
| (μg/g, | | | |
| garlic juice) | | | |

*Significant within 10%;
**significant within 1%.

### Example 3

### Comparison of Ajoene Production for Different Edible Oils

Peeled garlic (10 kg) was mashed using a mashing machine (Oscar, Kimhae, Korea) into garlic pulp and juice. Only the garlic juice was used for experiment. First, 5 g of the garlic juice was mixed well with 15 g of butter or edible oil (7 kinds) purchased from a local supermarket (Seongnam, Korea) and sonicated in a water bath of 40° C. for 20 minutes.

Ajoene preparation experiments were performed for different edible oils under the condition of reaction temperature=65° C., reaction time=2 hr and oil volume=3 times. As seen from Table 6, the ajoene content was 553-1301 μg/g (garlic juice). Butter showed about 2 times higher content than other edible oils, and there was no significant difference in ajoene production among the other edible oils except for butter.

TABLE 6
Comparison of ajoene content for different edible oils

| | Ajoene content | |
|---|---|---|
| Oils | (μg/g, garlic juice) | Z/E ratio |
| Butter | 1301^{a}* | 10.1 |
| Corn oil | 633^{b} | 8.2 |
| Olive oil | 656^{b} | 11.5 |
| Rice bran oil | 652^{b} | 13.1 |
| Grape seed oil | 604^{b} | 6.2 |
| Soybean oil | 672^{b} | 6.9 |
| Perilla oil | 553^{b} | 9.5 |
| Sesame oil | 608^{b} | 9.0 |

*Significance between butter^{a} and edible oils^{b} upon Duncan's multiple comparison test (P < 0.05)

The features and advantages of the present disclosure can be summarized as follows:

(a) The present disclosure provides an optimal condition for production of ajoene from garlic.

(b) According to the present disclosure, ajoene can be prepared with a yield 2 times or more than that of the existing methods. Thus, ajoene can be provided as active ingredient for foods and medicines more cost-effectively.

(c) The production protocol according to the present disclosure is suitable for large-scale production of ajoene.

While the present disclosure has been described with respect to the specific embodiments, it will be apparent to those skilled in the art that various changes and modifications may be made without departing from the spirit and scope of the disclosure as defined in the following claims.

1. A method for preparing ajoene comprising:
obtaining garlic pulp or garlic extract from garlic;
mixing the garlic pulp or garlic extract juice with animal lipid to form a reaction mixture; and
heating the reaction mixture to obtain ajoene.

2. The method according to claim 1, wherein the particle size of garlic pulp is 20 mesh or less and the garlic extract is garlic juice.

3. The method according to claim 1, wherein the animal lipid is animal fat or oil.

4. The method according to claim 3, wherein the animal lipid is animal fat.

5. The method according to claim 4, wherein the animal fat originates from the milk of cow, goat, sheep, horse, camel, donkey, deer, water buffalo or yak.

6. The method according to claim 5, wherein the animal fat originates from the milk of cow.

7. The method according to claim 1, wherein the weight ratio of the garlic juice to the animal lipid in the reaction mixture is 1:2-1:5.

8. The method according to claim 1, which further comprises sonicating the reaction mixture between said obtaining the garlic pulp or garlic extract juice and said mixing the garlic pulp or garlic extract with the animal lipid.

9. The method according to claim 1, wherein said heating is performed at 30-80° C.

10. The method according to claim 1, wherein said heating is performed for 1-5 hours.

11. The method according to claim 1, which further comprises, after said obtaining the ajoene, purifying and encapsulating the ajoene with gelatin.

12. Ajoene prepared by the method according to claim 1.

13. The ajoene according to claim 12, wherein the ajoene is encapsulated in a gelatin capsule.
